(19) United States
(12) Patent Application Publication
(10) Pub. No.: US 2026/0165984 A1
(43) Pub. Date: Jun. 18, 2026

(54) SELF-EMULSIFYING COMPOSITION OF COENZYME Q10 WITH HIGH BIOAVAILABILITY, AND METHOD AND DEVICE FOR PREPARING SAME

(71) Applicant: ALAND (HK) NUTRITION HOLDING LIMITED, Kowloon (HK)

(72) Inventors: Guodong ZHANG, Haining (CN); Jiemao SHI, Haining (CN); Gang LIU, Haining (CN); Zhongke CHEN, Haining (CN); Cheng CAO, Haining (CN)

(21) Appl. No.: 18/981,864

(22) Filed: Dec. 16, 2024

(51) Int. Cl. A61K 31/122; A61K 9/107; A61K 9/48; A61K 31/4745; A61K 47/10; A61K 47/14; A61K 47/22; A61K 47/24; A61K 47/26; A61K 47/44; B01F 23/41; B01F 23/43; B01F 27/805; B01F 27/90; B01F 35/32; B01F 35/50; B01F 35/90; B01F 35/92; B01F 101/22

(52) U.S. Cl. CPC A61K 31/122; A61K 9/107; A61K 9/4825; A61K 31/4745; A61K 47/10; A61K 47/14; A61K 47/22; A61K 47/24; A61K 47/26; A61K 47/44; B01F 23/4105; B01F 23/43; B01F 27/805; B01F 27/90; B01F 35/3204; B01F 35/514; B01F 35/92; B01F 2035/99; B01F 2101/22

(57) ABSTRACT

Disclosed are a self-emulsifying composition of a coenzyme Q10 with high bioavailability, and a method and device for preparing the same. The composition includes the following components in percentage by weight: 5-35% of a coenzyme Q10, 1-50% of an oil phase, 10-60% of a surfactant, 1-10% of a cosurfactant, 0-7% of an antioxidant, and 0.5-2% of pyrroloquinoline quinone disodium salt. The coenzyme Q10 medicinal liquid obtained can be kept in a clear and transparent solution state at room temperature, can be stably dispersed in water and self-emulsified to a transparent emulsion, significantly improves the bioavailability of the coenzyme Q10 by more than 4 times, and can be filled in capsules or softgels to swallow. The method disclosed by the disclosure includes two steps: material grinding and vacuum self-emulsifying. The method is concise in step and easy to control. Moreover, the device is simple in structure and low in cost.

### TECHNICAL FIELD

The disclosure relates to the technical field of healthcare foods, and particularly to a self-emulsifying composition of a coenzyme Q10 with high Bioavailability, and a method and device for preparing the same.

### BACKGROUND

The coenzyme Q10 exists in most eukaryocytes, particularly mitochondria, which is one of components of the respiratory chain, playing an important role in proton displacement and electron transfer in the in vivo respiratory chain. The coenzyme Q10 has strong antioxidant ability and can eliminate free radicals to prevent cells from being damaged and further can improve the myocardial function to protect nerve cells of the brain and enhance the vitality of the human body, so as to effectively prevent occurrence of cardiovascular and cerebrovascular diseases. The coenzyme Q10 can enhance the immunity of the human body, and as a non-specific immunopotentiator for the body, it may play a better role in improving the immunity of the body, resisting inflammation and tumors and the like. The coenzyme Q10 also has a certain positive effect on viral myocarditis, viral hepatitis, chronic hepatitis, diabetic neuritis, chronic obstructive pneumonia, bronchitis, asthma, periodontitis and the like. The coenzyme Q10 can alleviate some discomforts of cancer patients brought by radiotherapy, chemotherapy and the like.

The coenzyme Q10 needed by the human body is usually synthesized by the human body itself and is ingested from foods. With age, the content of the coenzyme Q10 synthesized by the human body itself is reduced, and therefore, the coenzyme Q10 needs to be supplemented by external foods and supplements. The coenzyme Q10 with large molecular weight is fat soluble, high in melting point, and insoluble in water. A common coenzyme Q10 oral preparation is hardly absorbed by the human body. Being insoluble in water, it is hardly digested and absorbed in the human body. Increasing the solubility of the coenzyme Q10 in water by using a self-emulsifying system to improve the bioavailability in digestion and absorption by the human body becomes a new research direction.

### SUMMARY

To overcome the deficiencies in the prior art, the disclosure provides a self-emulsifying composition of a coenzyme Q10 with high bioavailability. The composition can not only improve the solubility of the coenzyme Q10 in water and improve the bioavailability in digestion and absorption by the human body, but also improve the energy level of human cells.

The disclosure is achieved by the following technical solution:

A self-emulsifying composition of a coenzyme Q10 with high bioavailability, including the following components in percentage by weight: 5-35% of a coenzyme Q10, 1-50% of an oil phase, 10-60% of a surfactant, 1-10% of a cosurfactant, 0-7% of an antioxidant, and 0.5-2% of pyrroloquinoline quinone disodium salt.

The self-emulsifying composition includes the following components in percentage by weight: 8-18% of a coenzyme Q10, 25-45% of an oil phase, 30-55% of a surfactant, 1-5% of a cosurfactant, 1-7% of an antioxidant, and 0.5-1.5% of pyrroloquinoline quinone disodium salt.

The self-emulsifying composition includes the following components in percentage by weight: 10-15% of a coenzyme Q10, 31-35% of an oil phase, 36-44% of a surfactant, 1-3% of a cosurfactant, 1-6% of an antioxidant, and 0.5-1% of pyrroloquinoline quinone disodium salt.

The oil phase is one or more of fish oil, medium chain triglyceride, soybean oil, olive oil, sunflower seed oil, and corn oil.

The surfactant is selected from one or more of Tween80, phospholipid, polyglycerol fatty acid ester, and mono-diglyceride.

The cosurfactant is selected from one or more of glycerinum, polyethylene glycol, and a sorbitol solution.

The antioxidant is selected from one or more of vitamin E, ascorbic acid, natural vitamin E and ester derivatives thereof, and rosemary extract.

The coenzyme Q10 is at least one of an oxidized coenzyme Q10 and a reduced coenzyme Q10.

In addition, the disclosure further provides a device for preparing a self-emulsifying composition of a coenzyme Q10 with high bioavailability, including a multi-roller grinder and an emulsifying vacuum tank, where
the multi-roller grinder is configured to grind a material of the self-emulsifying composition of the coenzyme Q10 and includes a frame body, a plurality of rollers are rotationally mounted on the frame body, a plurality of motors are mounted on the frame body, the quantity of the plurality of motors is consistent with the quantity of the plurality of rollers, and when the plurality of motors run, the motors drive corresponding rollers to rotate respectively and enable the plurality of rollers to perform non-synchronous running to grind the material of the self-emulsifying composition of the coenzyme Q10; the multi-roller grinder further includes a shoveling apparatus movably arranged on the frame body, and the shoveling apparatus can be in contact with the rotating rollers to shovel the material attached to the peripheral surfaces of the rollers; and
the emulsifying vacuum tank is configured to self-emulsify the ground material of the self-emulsifying composition of the coenzyme Q10 and includes a fixed frame, a seal cover is mounted in a penetrating manner on the top surface of the fixed frame, a driving motor is mounted on the seal cover, a shaft lever is mounted on a power output shaft of the driving motor, a stirring rod is mounted on the shaft lever, a transparent tank is slidably arranged in the fixed frame, the top of the transparent tank is opened, and the transparent tank is in insertion fit with the seal cover.

The shoveling apparatus includes a shovel blade, and the shovel blade is provided with a vertical part and an inclined part; a threaded barrel and a threaded rod in threaded connection to the threaded barrel are mounted on the vertical part, and a turnplate is mounted at an end of the threaded rod away from the frame body.

The shoveling apparatus is further provided with two guide bars, and the two guide bars are fixedly arranged on the side surface of the frame body and penetrate through the vertical part.

A multi-stage hydraulic cylinder is mounted on the inner bottom surface of the fixed frame, a push plate is mounted at an extension end of the multi-stage hydraulic cylinder, and the push plate is fixedly connected to the bottom surface of the transparent tank.

Sliding grooves are formed on inner walls of both sides of the fixed frame, a pair of slide bars is in pivoted connection to both sides of the transparent tank, and the end of each of the slide bars is movably received in the corresponding one of the sliding grooves.

A vacuum pump and a gas-pressure meter are mounted on the top surface of the seal cover, an air extraction end of the vacuum pump extends to the lower side of the seal cover, and a detection end of the gas-pressure meter also extends to the lower side of the seal cover; a gas container is further mounted on the top surface of the seal cover, the gas container is configured to store an inert gas, a gas delivery pipe is mounted on the gas container, the gas delivery pipe extends to the lower side of the seal cover, and a switching valve is mounted on the gas delivery pipe.

Finally, the disclosure further provides a method for preparing the self-emulsifying composition of a coenzyme Q10 with high bioavailability, including the following steps:
(1) material grinding: inputting a material of a coenzyme Q10 into the multi-roller grinder for grinding and refining;
(2) preparation of a coenzyme Q10 medicinal liquid: putting the ground coenzyme Q10 and an oil phase material at a certain ratio into the emulsifying vacuum tank to be uniformly mixed, heating the mixture to 45-60° C., stirring the mixture till the coenzyme Q10 is completely dissolved, and adding the surfactant and the cosurfactant at a corresponding ratio to obtain the coenzyme Q10 medicinal liquid; and
(3) preparation of a coenzyme Q10 preparation: further preparing the coenzyme Q10 medicinal liquid prepared in step (2) into any one of coenzyme Q10 softgels, coenzyme Q10 liquid hard capsules, a coenzyme Q10 oral solution, and coenzyme Q10 drops.

Use of the self-emulsifying composition of a coenzyme Q10 with high bioavailability in preparation of foods, cosmetics, healthcare products or drugs.

Compared with the prior art, the disclosure has the following advantages:

1. The self-emulsifying preparation of the coenzyme Q10 with high bioavailability prepared from the above components using the above method can be kept in a clear and transparent liquid state at room temperature and can be dispersed in water to form a self-emulsifying transparent nano emulsion, with the particle size being 1-100 nm, so that the self-emulsifying effect is guaranteed and the bioavailability is improved. Compared with the commercial oxidized coenzyme Q10, the bioavailability of the coenzyme Q10 preparation with high bioavailability provided by the disclosure is improved by 4 times.

2. Pyrroloquinoline quinone disodium salt (PQQ for short) in a certain weight percent is added into the coenzyme Q10 composition with high bioavailability provided by the disclosure, which is a potent antioxidant to stimulate generation of new mitochondria and increase the efficiency of cellular energy metabolism, so as to have a significant synergistic effect of jointly improving the energy level of cells, the cardiovascular health and the function of mitochondria with the coenzyme Q10 in the composition.

3. The coenzyme Q10 composition with high bioavailability provided by the disclosure uses vitamin E or a composition thereof in a certain weight percent, which protects, through its antioxidative effect, the coenzyme Q10 from being damaged by oxidative stress and also helps its absorption in the intestinal tract.

4. In the device for preparing the self-emulsifying solution of the coenzyme Q10 provided by the disclosure, the multi-roller grinder is provided with the plurality of motors to drive the plurality of rollers to rotate to perform non-synchronous running to achieve a controllable and sufficient grinding effect through extruding friction on the surfaces of the rollers at different relative speeds, so that the mixed material liquid is finer and smoother. Moreover, after grinding is completed, the shoveling apparatus is in contact with the rollers to shovel the mixed material liquid on the rollers, so that the loss of the mixed material liquid is reduced and the cleaning difficulty of the multi-roller grinder is reduced.

5. In the device for preparing the self-emulsifying solution of the coenzyme Q10 provided by the disclosure, the emulsifying vacuum tank is provided with the multi-stage hydraulic cylinder to adjust the height of the transparent tank through the push plate during running. The transparent tank is rotationally arranged, so that it is convenient to pour out the material in the transparent tank. By starting the vacuum pump to pump out air in the transparent tank, the interior of the transparent tank is in a negative pressure state. After the inert gas is introduced via the gas delivery pipe, the self-emulsifying solution is not easily oxidized.

6. Through the above preparation method and device provided by the disclosure, the device is simple in structure, the preparation method is concise in step and easy to control, industrial production is convenient to realize, and the method is economic and environmentally friendly.

### BRIEF DESCRIPTION OF FIGURES

FIG. 1 is a schematic diagram of particle size distribution of a self-emulsifying preparation of a coenzyme Q10 after emulsification;

FIG. 2 is a schematic diagram of a drug concentration-time curve of the coenzyme Q10;

FIG. 3 is a pseudo-ternary phase diagram of the coenzyme Q10 provided by the disclosure in different accessories;

FIG. 4 is a three-dimensional assembly diagram of a multi-roller grinder in a preparation device for a self-emulsifying composition of the coenzyme Q10 disclosed by the disclosure;

FIG. 5 is another schematic assembly diagram of the multi-roller grinder in FIG. 4;

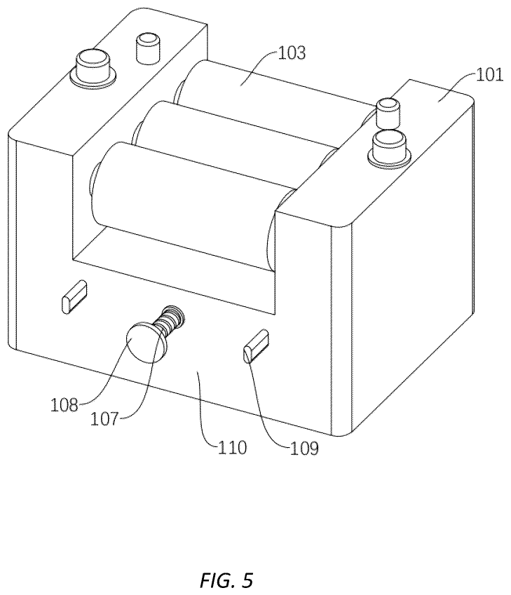

FIG. 6 is a partially assembly schematic structural diagram of the multi-roller grinder in FIG. 4;

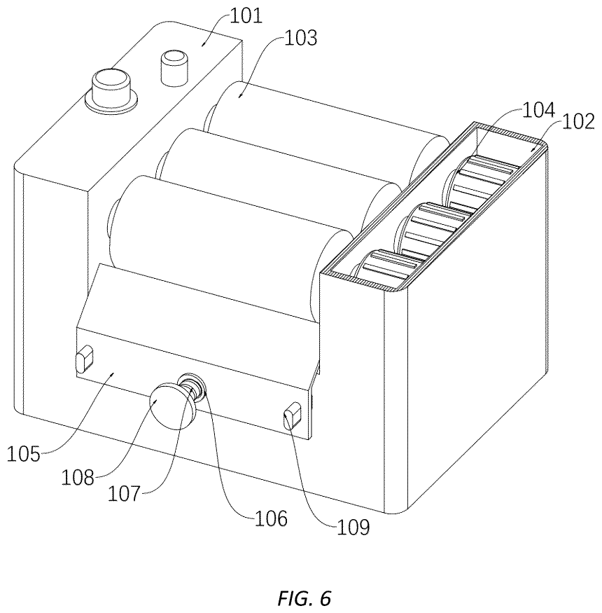

FIG. 7 is a partial three-dimensional diagram of a shoveling apparatus in the disclosure;

FIG. 8 is a right view of FIG. 7;

FIG. 9 is a three-dimensional diagram of an emulsifying vacuum tank in the preparation device for a self-emulsifying composition of the coenzyme Q10 disclosed by the disclosure; and

FIG. 10 is a three-dimensional diagram of the emulsifying vacuum tank in FIG. 9 at another angle.

### DETAILED DESCRIPTION

To make objectives, technical solutions and advantages of the present disclosure more clear, the present disclosure is described in detail below in conjunction with embodiments, so that those skilled in the art can fully understand the technical content of the present disclosure. It should be understood that the embodiments below are used to further describe the present disclosure and cannot be construed as a limitation to the scope of protection of the disclosure. Some nonessential improvements and adjustments made by those skilled in the art based on the above content of the disclosure should all fall within the scope of protection of the disclosure. Specific preparation method parameters of the examples below are merely an example in an appropriate range, i.e., those skilled in the art can make choice in the appropriate range through the description of the disclosure, rather than being limited to the specific numerical values of the examples below.

A self-emulsifying composition of a coenzyme Q10 with high bioavailability provided by the disclosure includes the following components in percentage by weight: 5-35% of a coenzyme Q10, 1-50% of an oil phase, 10-60% of a surfactant, 1-10% of a cosurfactant, 0-7% of an antioxidant, and 0.5-2% of pyrroloquinoline quinone disodium salt.

A self-emulsifying composition of a coenzyme Q10 with high bioavailability includes the following components in percentage by weight: 8-18% of a coenzyme Q10, 25-45% of an oil phase, 30-55% of a surfactant, 1-5% of a cosurfactant, 0-6% of an antioxidant, and 0.5-1.5% of pyrroloquinoline quinone disodium salt.

A self-emulsifying composition of a coenzyme Q10 with high bioavailability includes the following components in percentage by weight: 10-15% of a coenzyme Q10, 31-35% of an oil phase, 36-44% of a surfactant, 1-3% of a cosurfactant, 0-5% of an antioxidant, and 0.5-1% of pyrroloquinoline quinone disodium salt.

The oil phase is one or more of fish oil, medium chain triglyceride, soybean oil, olive oil, sunflower seed oil, and corn oil.

The surfactant is selected from one or more of Tween80, phospholipid, polyglycerol fatty acid ester, and mono-diglyceride.

The cosurfactant is selected from one or more of glycerinum, polyethylene glycol, and a sorbitol solution.

The disclosure further provides use of the self-emulsifying composition of a coenzyme Q10 with high bioavailability in preparation of foods, cosmetics, healthcare products or drugs.

The concept of the disclosure is further illustrated and described below in conjunction with specific embodiments, which does not mean that the disclosure is merely limited to the specific solutions described below. The value in the embodiments is any specific numerical value within the disclosure, which is implementable.

### Example 1

A self-emulsifying composition of a coenzyme Q10 with high bioavailability includes the following components in percentage by weight: 5% of a coenzyme Q10, 47% of medium chain triglyceride, 37% of mono-diglyceride, 1% of pyrroloquinoline quinone disodium salt, and 10% of glycerinum.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and medium chain triglyceride in a corresponding weight percent were inputted into an emulsifying vacuum tank to be heated to 45-60° C., the mixture was insulated and stirred till the coenzyme Q10 was completely dissolved, mono-diglyceride was added, then glycerinum was then added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 50 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 2

A self-emulsifying composition of a coenzyme Q10 with high bioavailability includes the following components in percentage by weight: 10% of a coenzyme Q10, 42.5% of fish oil, 5% of vitamin E, 37% of mono-diglyceride, 0.5% of pyrroloquinoline quinone disodium salt, and 5% of glycerinum.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and fish oil and vitamin E in corresponding weight percents were inputted into an emulsifying vacuum tank to be heated to 45-55° C., the mixture was insulated and stirred till the coenzyme Q10 was completely dissolved, mono-diglyceride was added, then glycerinum was then added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 100 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 3

A self-emulsifying composition of a coenzyme Q10 with high bioavailability includes the following components in percentage by weight: 20% of a coenzyme Q10, 19.8% of medium chain triglyceride, 54% of polyglycerol fatty acid ester, 1.2% of pyrroloquinoline quinone disodium salt, and 5% of glycerinum.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and medium chain triglyceride in a corresponding weight percent were inputted into an emulsifying vacuum tank to be heated to 55° C., the mixture was insulated and stirred till the coenzyme Q10 was completely dissolved, polyglycerol fatty acid ester was added, then glycerinum was then added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 200 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 4

A self-emulsifying composition of a coenzyme Q10 with high bioavailability includes the following components in percentage by weight: 35% of a coenzyme Q10, 30.2% of medium chain triglyceride, 31% of Tween80, 3% of glycerinum, and 0.8% of pyrroloquinoline quinone disodium salt.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and medium chain triglyceride in a corresponding weight percent were inputted into an emulsifying vacuum tank to be heated to 55° C., the mixture was insulated and stirred till the coenzyme Q10 was completely dissolved, Tween80 was added, then glycerinum was then added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 350 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 5

A self-emulsifying composition of a coenzyme Q10 with high bioavailability includes the following components in percentage by weight: 18% of a coenzyme Q10, 49% of sunflower seed oil, 30% of mono-diglyceride, 1% of pyrroloquinoline quinone disodium salt, and 2% of glycerinum.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and sunflower seed oil and vitamin E in corresponding weight percents were inputted into an emulsifying vacuum tank to be heated to 55° C., the mixture was insulated and stirred till the coenzyme Q10 was completely dissolved, mono-diglyceride was added, then glycerinum was then added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 80 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 6

A self-emulsifying composition of a coenzyme Q10 with high bioavailability includes the following components in percentage by weight: 5% of a coenzyme Q10, 3% of vitamin E, 30.5% of soybean oil, 24% of Tween80, 7% of phospholipid, 26% of polyglycerol fatty acid ester, 0.5% of pyrroloquinoline quinone disodium salt, and 4% of glycerinum.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and soybean oil and vitamin E in corresponding weight percents were inputted into an emulsifying vacuum tank to be heated to 45-55° C., the mixture was insulated and stirred till the coenzyme Q10 was completely dissolved, phospholipid, Tween80, and polyglycerol fatty acid ester were added, finally, glycerinum was added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 50 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 7

A self-emulsifying composition of a coenzyme Q10 with high bioavailability includes the following components in percentage by weight: 15% of a coenzyme Q10, 33.5% of medium chain triglyceride, 10% of Tween80, 7% of phospholipid, 28% of polyglycerol fatty acid ester, 1.5% of pyrroloquinoline quinone disodium salt, and 5% of glycerinum.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and medium chain triglyceride in a corresponding weight percent were inputted into an emulsifying vacuum tank to be heated to 45-55° C., the mixture was insulated and stirred till the coenzyme Q10 was completely dissolved, phospholipid, Tween80, and polyglycerol fatty acid ester were added; then glycerinum was added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 150 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 8

A self-emulsifying composition of a coenzyme Q10 with high bioavailability includes the following components in percentage by weight: 10% of a coenzyme Q10, 5% of vitamin E, 34% of fish oil, 9% of polyglycerol fatty acid ester, 9% of phospholipid, 29% of Tween 80, 1% of pyrroloquinoline quinone disodium salt, and 3% of glycerinum.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and fish oil and vitamin E in corresponding weight percents were inputted into an emulsifying vacuum tank to be heated to 45-55° C., the mixture was insulated and stirred till the coenzyme Q10 was completely dissolved, and Tween80, polyglycerol fatty acid ester, and phospholipid were added; then glycerinum was added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 100 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 9

A self-emulsifying composition of a coenzyme Q10 with high bioavailability includes the following components in percentage by weight: 8% of a coenzyme Q10, 5% of vitamin E, 14% of fish oil, 34.1% of medium chain triglyceride, 6% of Tween80, 19% of polyglycerol fatty acid ester, 5% of mono-diglyceride, 5% of phospholipid, 3% of glycerinum, and 0.9% of pyrroloquinoline quinone disodium salt.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and fish oil, vitamin E, and medium chain triglyceride in corresponding weight percents were inputted into an emulsifying vacuum tank to be heated to 45-55° C., the mixture was insulated and stirred till the coenzyme Q10 was completely dissolved, Tween80, polyglycerol fatty acid ester, mono-diglyceride, and phospholipid were added, then glycerinum was added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 80 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Example 10

A self-emulsifying composition of a coenzyme Q10 with high bioavailability includes the following components in percentage by weight: 18% of a coenzyme Q10, 5% of vitamin E, 25.2% of medium chain triglyceride, 10% of Tween80, 25% of polyglycerol fatty acid ester, 6% of phospholipid, 6% of mono-diglyceride, 4% of glycerinum, and 0.8% of pyrroloquinoline quinone disodium salt.

A preparation method includes the following steps:
(1) material grinding: a material of a coenzyme Q10 was inputted into the multi-roller grinder for grinding and refining;
(2) preparation of a coenzyme Q10 medicinal liquid: the ground coenzyme Q10 and medium chain triglyceride and vitamin E in corresponding weight percents were inputted into an emulsifying vacuum tank to be heated to 45-55° C., the mixture was insulated and stirred till the coenzyme Q10 was completely dissolved, and Tween80, mono-diglyceride, polyglycerol fatty acid ester, and phospholipid were added; and then glycerinum was added, and the mixture was uniformly mixed, where the whole system was an orange red, uniform and transparent liquid to obtain the coenzyme Q10 medicinal liquid; and
(3) preparation of a coenzyme Q10 preparation: the medicinal liquid was taken as a content of softgels to prepare softgels, where the content of each softgel was 1000 mg, and the content of the coenzyme Q10 was 180 mg.

Self-emulsifying detection: 1 g of the product was added into 100 ml of purified water, and the mixture was slightly oscillated to obtain an orange-yellow transparent aqueous solution. It could be seen that the product was good in water dispersibility.

### Comparative Example 1

A self-emulsifying composition of a coenzyme Q10 with high bioavailability includes the following components in percentage by weight: 5% of a coenzyme Q10 and 95% of soybean oil.

A preparation method includes the following steps:
(1) preparation of a coenzyme Q10 medicinal liquid: the soybean oil and the coenzyme Q10 at prescription dosages were heated to 45-55° C., and the mixture was insulated and stirred till the coenzyme Q10 was completely dissolved, where the whole system was an orange red, uniform and transparent liquid to obtain the coenzyme Q10 medicinal liquid; and
(2) preparation of a coenzyme Q10 preparation: the medicinal liquid was taken as a content of softgels to prepare softgels, where the content of each softgel was 1000 mg, and the content of the coenzyme Q10 was 50 mg.

Self-emulsifying detection: 1 g of the product was added into 100 ml of purified water, and the mixture was severely oscillated and placed to layer immediately, where the oily content floated on water, so that no orange-yellow transparent emulsion could be obtained. It could be seen that comparative example 1 could not self-emulsify in water.

### Comparative Example 2

A self-emulsifying composition of a coenzyme Q10 with high bioavailability includes the following components in percentage by weight: 8% of a coenzyme Q10 and 92% of olive oil.

A preparation method includes the following steps:
(1) preparation of a coenzyme Q10 medicinal liquid: the olive oil and the coenzyme Q10 at prescription dosages were heated to 45-55° C., and the mixture was insulated and stirred till the coenzyme Q10 was completely dissolved, where the whole system was an orange red, uniform and transparent liquid to obtain the coenzyme Q10 medicinal liquid; and
(2) preparation of a coenzyme Q10 preparation: the medicinal liquid was taken as a content of softgels to prepare softgels, where the content of each softgel was 1000 mg, and the content of the coenzyme Q10 was 80 mg.

Self-emulsifying detection: 1 g of the product was added into 100 ml of purified water, and the mixture was severely oscillated and placed to layer immediately, where the oily content floated on water, so that no orange-yellow transparent emulsion could be obtained. It could be seen that comparative example 2 could not self-emulsify in water.

### Comparative Example 3

A self-emulsifying composition of a coenzyme Q10 with high bioavailability includes the following components in percentage by weight: 5% of a coenzyme Q10, 5% of vitamin E, 20% of fish oil, 40% of medium chain triglyceride, 10% of mono-diglyceride, and 20% of polyglycerol fatty acid ester.

A preparation method includes the following steps:
(1) preparation of a coenzyme Q10 medicinal liquid: the olive oil, medium chain triglyceride, vitamin E, and the coenzyme Q10 at prescription dosages were heated to 45-55° C., the mixture was insulated and stirred till the coenzyme Q10 was completely dissolved, and mono-diglyceryl fatty acid esters and polyglycerol fatty acid ester were added; the whole system was an orange red, uniform and transparent liquid to obtain the coenzyme Q10 medicinal liquid; and
(2) preparation of a coenzyme Q10 preparation: the medicinal liquid was taken as a content of softgels to prepare softgels, where the content of each softgel was 1000 mg, and the content of the coenzyme Q10 was 50 mg.

Self-emulsifying detection: 1 g of the product was added into 100 ml of purified water, and the mixture was severely oscillated and placed to layer immediately, where the oily content floated on water, so that no orange-yellow transparent emulsion could be obtained. It could be seen that comparative example 3 could not self-emulsify in water.

### Comparative Example 4

A self-emulsifying composition of a coenzyme Q10 with high bioavailability includes the following components in percentage by weight: 15% of a coenzyme Q10, 5% of vitamin E, 24% of fish oil, 31% of medium chain triglyceride, 5% of mono-diglyceride, 10% of polyglycerol fatty acid ester, 5% of Tween80, and 5% of glycerinum.

A preparation method includes the following steps:
(1) preparation of a coenzyme Q10 medicinal liquid: fish oil, medium chain triglyceride, vitamin E, and the coenzyme Q10 at prescription dosages were heated to 45-55° C., the mixture was insulated and stirred till the coenzyme Q10 was completely dissolved, mono-diglyceride, Tween80, and polyglycerol fatty acid ester were added, and then glycerinum was added, and the mixture was uniformly mixed, where the whole system was an orange red, uniform and transparent liquid to obtain the coenzyme Q10 medicinal liquid; and
(2) preparation of a coenzyme Q10 preparation: the medicinal liquid was taken as a content of softgels to prepare softgels, where the content of each softgel was 1000 mg, and the content of the coenzyme Q10 was 150 mg.

Self-emulsifying detection: 1 g of the product was added into 100 ml of purified water, and the mixture was severely oscillated and placed to layer immediately, where the oily content floated on water, so that no orange-yellow transparent emulsion could be obtained. It could be seen that comparative example 4 could not self-emulsify in water.

### Comparative Example 5

A self-emulsifying composition of a coenzyme Q10 with high bioavailability includes the following components in percentage by weight: 5% of a coenzyme Q10, 1% of vitamin E, 22% of fish oil, 30% of medium chain triglyceride, and 42% of polyglycerol fatty acid ester.

A preparation method includes the following steps:
(1) preparation of a coenzyme Q10 medicinal liquid: fish oil, vitamin E, medium chain triglyceride, and the coenzyme Q10 at prescription dosages were heated to 45-55° C., the mixture was insulated and stirred till the coenzyme Q10 was completely dissolved, and polyglycerol fatty acid ester was added, where the whole system was an orange red, uniform and transparent liquid to obtain the coenzyme Q10 medicinal liquid; and
(2) preparation of a coenzyme Q10 preparation: the medicinal liquid was taken as a content of softgels to prepare softgels, where the content of each softgel was 1000 mg, and the content of the coenzyme Q10 was 50 mg.

Self-emulsifying detection: 1 g of the product was added into 100 ml of purified water, and the mixture was severely oscillated and placed to layer immediately, where the oily content floated on water, so that no orange-yellow transparent emulsion could be obtained. It could be seen that comparative example 5 could not self-emulsify in water.

### Comparative Example 6

A self-emulsifying composition of a coenzyme Q10 with high bioavailability includes the following components in percentage by weight: 5% of a coenzyme Q10, 1% of vitamin E, 19% of fish oil, 35% of medium chain triglyceride, 6% of Tween80, 30% of polyglycerol fatty acid ester, and 4% of glycerinum.

A preparation method includes the following steps:
(1) preparation of a coenzyme Q10 medicinal liquid: fish oil, vitamin E, medium chain triglyceride, and the coenzyme Q10 at prescription dosages were heated to 45-55° C., the mixture was insulated and stirred till the coenzyme Q10 was completely dissolved, Tween80 and polyglycerol fatty acid ester were added, then glycerinum was added, and the mixture was uniformly mixed, where the whole system was an orange red, uniform and transparent liquid to obtain the coenzyme Q10 medicinal liquid; and
(2) preparation of a coenzyme Q10 preparation: the medicinal liquid was taken as a content of softgels to prepare softgels, where the content of each softgel was 1000 mg, and the content of the coenzyme Q10 was 50 mg.

Self-emulsifying detection: 1 g of the product was added into 100 ml of purified water, and the mixture was severely oscillated and placed to layer immediately, where the oily content floated on water, so that no orange-yellow transparent emulsion could be obtained. It could be seen that comparative example 6 could not self-emulsify in water.

### Comparative Example 7

A self-emulsifying composition of a coenzyme Q10 with high bioavailability includes the following components in percentage by weight: 15% of a coenzyme Q10, 5% of vitamin E, 14% of fish oil, 39% of medium chain triglyceride, 3% of phospholipid, 20% of polyglycerol fatty acid ester, and 4% of glycerinum.

A preparation method includes the following steps:
(1) preparation of a coenzyme Q10 medicinal liquid: fish oil, vitamin E, medium chain triglyceride, and the coenzyme Q10 at prescription dosages were heated to 45-55° C., the mixture was insulated and stirred till the coenzyme Q10 was completely dissolved, polyglycerol fatty acid ester and phospholipid were added, then glycerinum was added, and the mixture was uniformly mixed, where the whole system was an orange red, uniform and transparent liquid to obtain the coenzyme Q10 medicinal liquid; and
(2) preparation of a coenzyme Q10 preparation: the medicinal liquid was taken as a content of softgels to prepare softgels, where the content of each softgel was 1000 mg, and the content of the coenzyme Q10 was 150 mg.

Self-emulsifying detection: 1 g of the product was added into 100 ml of purified water, and the mixture was severely oscillated and placed to layer immediately, where the oily content floated on water, so that no orange-yellow transparent emulsion could be obtained. It could be seen that comparative example 7 could not self-emulsify in water.

The self-emulsifying preparations of the coenzyme Q10 prepared in the above examples 1-10 and comparative examples 1-7 were subjected to stability experiments to observe whether layered, turbid, and precipitated phenomena appear at different temperatures. The self-emulsifying preparations of the coenzyme Q10 were stored in environments at 10° C., 25° C., and 37° C. for one week. The samples were subjected to visual detection to detect the stability of the samples. See Table 1.

TABLE 1
Performance of different self-emulsifying preparations
of coenzyme Q10 at different temperatures

| | Observe after being stored for one week | | | |
|---|---|---|---|---|
| Examples | Initial state | 10° C. | 25° C. | 37° C. |
| Example 1 | Orange-red transparent | No | No | No |
| | and uniform liquid | remarkable | remarkable | remarkable |
| | | changes | changes | changes |
| Example 2 | Orange-red transparent | No | No | No |
| | and uniform liquid | remarkable | remarkable | remarkable |
| | | changes | changes | changes |
| Example 3 | Orange-red transparent | No | No | No |
| | and uniform liquid | remarkable | remarkable | remarkable |
| | | changes | changes | changes |
| Example 4 | Orange-red transparent | No | No | No |
| | and uniform liquid | remarkable | remarkable | remarkable |
| | | changes | changes | changes |
| Example 5 | Orange-red transparent | No | No | No |
| | and uniform liquid | remarkable | remarkable | remarkable |
| | | changes | changes | changes |
| Example 6 | Orange-red transparent | No | No | No |
| | and uniform liquid | remarkable | remarkable | remarkable |
| | | changes | changes | changes |
| Example 7 | Orange-red transparent | No | No | No |
| | and uniform liquid | remarkable | remarkable | remarkable |
| | | changes | changes | changes |
| Example 8 | Orange-red transparent | No | No | No |
| | and uniform liquid | remarkable | remarkable | remarkable |
| | | changes | changes | changes |
| Example 9 | Orange-red transparent | No | No | No |
| | and uniform liquid | remarkable | remarkable | remarkable |
| | | changes | changes | changes |
| Example 10 | Orange-red transparent | No | No | No |
| | and uniform liquid | remarkable | remarkable | remarkable |
| | | changes | changes | changes |
| Comparative | Orange-red transparent | Orange- | Orange-yellow | Orange-yellow |
| example 1 | and uniform liquid | yellow solid | semi-solid | semi-solid |
| Comparative | Orange-red transparent | Orange- | Orange-yellow | Orange-yellow |
| example 2 | and uniform liquid | yellow solid | solid | solid |
| Comparative | Orange-red transparent | Orange- | Orange-yellow | Orange-yellow |
| example 3 | and uniform liquid | yellow solid | semi-solid | semi-solid |
| Comparative | Orange-red transparent | Orange- | Orange-yellow | Orange-yellow |
| example 4 | and uniform liquid | yellow solid | semi-solid | solid |
| Comparative | Orange-red transparent | Orange- | Orange-yellow | Orange-yellow |
| example 5 | and uniform liquid | yellow semi- | semi-solid | semi-solid |
| | | solid | | |
| Comparative | Orange-red transparent | Orange- | Orange-yellow | Orange-yellow |
| example 6 | and uniform liquid | yellow solid | semi-solid | semi-solid |
| Comparative | Orange-red transparent | Orange- | Orange-yellow | Orange-yellow |
| example 7 | and uniform liquid | yellow solid | semi-solid | semi-solid |

The stability experiment result of the above table shows that the self-emulsifying preparations of the coenzyme Q10 obtained by the disclosure can keep good stability of the system in relatively low temperature and high temperature ranges without split-phase and precipitated phenomena.

After the self-emulsifying preparations of the coenzyme Q10 prepared in examples 1-10 and comparative examples 1-7 were stored at 25° C. for one week, 1 g of samples were taken and added to 100 ml of purified water to oscillate to observe the dispersibility in water; the quantity of heat production of oxidized nutrients (sugar, fat, and proteins) of the body within a certain time was determined by an indirect calorimetric method, which was the energy metabolic rate representing changes of the energy level; through mouse experiments, the concentration of the coenzyme Q10 in blood within the same time was determined to represent the intestinal absorption level, specifically shown in table 2.

TABLE 2
Dispersibility, energy level, and intestinal absorption
level of different self-emulsifying preparations
of the coenzyme Q10 in purified water

| | | | Intestinal |
|---|---|---|---|
| | | Energy | absorption |
| Examples | Dispersibility in water | level | level |
| Example 1 | Transparent and uniform liquid | Higher | Stronger |
| Example 2 | Transparent and uniform liquid | Higher | Stronger |
| Example 3 | Transparent and uniform liquid | Higher | Stronger |
| Example 4 | Transparent and uniform liquid | Higher | Stronger |
| Example 5 | Transparent and uniform liquid | High | Strong |
| Example 6 | Transparent and uniform liquid | High | Strong |
| Example 7 | Transparent and uniform liquid | Higher | Stronger |
| Example 8 | Transparent and uniform liquid | High | Strong |
| Example 9 | Transparent and uniform liquid | High | Strong |
| Example 10 | Transparent and uniform liquid | High | Strong |
| Comparative | Oily flotage | Low | Weak |
| example 1 | | | |
| Comparative | Oily flotage | Low | Weak |
| example 2 | | | |
| Comparative | Oily flotage | Lower | Weaker |
| example 3 | | | |
| Comparative | Oily flotage, solid precipitate | Lower | Weaker |
| example 4 | | | |
| Comparative | Oily flotage | Low | Weak |
| example 5 | | | |
| Comparative | Oily flotage | Low | Weak |
| example 6 | | | |
| Comparative | Oily flotage, solid precipitate | Lower | Weaker |
| example 7 | | | |

The experimental results of the above table 2 show the following:

1. The self-emulsifying softgels of the coenzyme Q10 obtained by the disclosure can self-emulsify in water and has good dispersibility.

2. Pyrroloquinoline quinone disodium salt in a certain weight percent is added into self-emulsifying preparation of the coenzyme Q10 provided by the disclosure, which is a potent antioxidant to stimulate generation of new mitochondria and increase the efficiency of cellular energy metabolism, so as to have a significant synergistic effect of jointly improving the energy level of cells, the cardiovascular health and the function of mitochondria with the coenzyme Q10 in the composition.

3. The coenzyme Q10 composition with high bioavailability provided by the disclosure uses the vitamin E composition in a certain weight percent, which protects, through its antioxidative effect, the coenzyme Q10 from being damaged by oxidative stress and also helps its absorption in the intestinal tract.

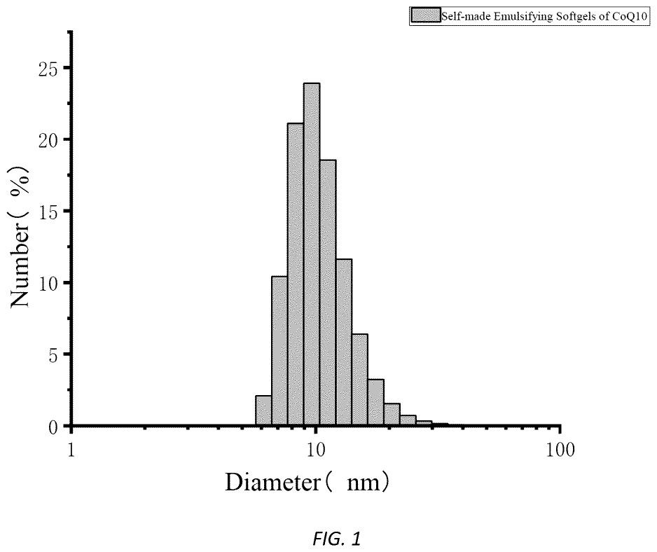

As shown in FIG. 1, the particle size range of the self-microemulsion of the emulsified coenzyme Q10 determined by a particle size potentiometer is 1-100 nm.

An oral bioavailability experiment of a rat is specifically as follows:

Verified by the animal experiment, the self-emulsifying solution of the coenzyme Q10 can significantly improve the in vivo bioavailability of the coenzyme Q10, with the test steps and results as follows:

Samples: the self-emulsifying softgels of the coenzyme Q10 prepared in example V of the disclosure; reference substances: commercially available oxidized coenzyme Q10 softgels and commercially available reduced coenzyme Q10 softgels, respectively.

Experimental steps are as follows:

Step 1: Establishment of an in vivo analytical method of the coenzyme Q10.

The content of the coenzyme Q10 in plasma was determined by high performance liquid chromatography. Chromatographic conditions are as follows:
Chromatographic column: ZORBAX Eclipse XDB-C18 (250 mm×4.6 mm, 5 μm);
Moving phase: methanol-anhydrous ethanol=50-50;
Column temperature: 35° C.;
Detection wavelength: 275 nm;
Flow rate: 1 ml/min.

Step 2: Plasma sample treatment method: 200 μL of a plasma sample was taken and 800 μL of methanol was added to vortex for 1 min. 1 mL of n-hexane was added to vortex for 3 min, and the mixture was centrifuged at 2000 rpm for 10 min. The organic layer was transferred to a novel centrifuge tube, then another 1 mL of n-hexane was added to an aqueous layer to vortex for 3 min, and the mixture was centrifuged at 4000 rpm for 10 min, and the organic layers were combined. The organic layer was blow-dried with high purity nitrogen at 50° C. in a water bath condition. 100 μL of ethanol was added to the centrifuge tube to redissolve and vortex for 5 min. At 4° C., the mixture was centrifuged at 14000 rpm for 15 min, and 50 μL of the mixture was taken for sample injection analysis.

Step 3: Drawing of a plasma sample standard curve.

10 mg of the coenzyme Q10 was precisely weighed, placed in a 50 mL volumetric flask, and dissolved with ethanol and volume-metered. A series of coenzyme Q10 standard solutions with the final concentrations of 2, 4, 6, 8, 12.8, 16 and 20 μg/mL were diluted with ethanol.

10 μL coenzyme Q10 standard solutions with different concentrations were taken, and 200 μL of blank plasma was added to the solutions to vortex and mix the solutions for 1 min as plasma standard curve samples with different concentrations. Treated by the “plasma sample treatment method” under the item 2.2, the solutions were determined. Peak areas of the drug at different concentrations were recorded, and a standard curve between the peak areas and the plasma drug concentrations was drawn.

Step 4: Administration regimen for animals.

The dosage for adults (60 kg) per day was determined as 100 mg according to the dosage of the commercially available coenzyme Q10. The administration dosage for rats was determined as 10 mg/kg according to the administration dosage conversion method between humans and rats reported in literatures.

18 male SD rats were taken and randomly divided into three groups, with 6 rats in each group.

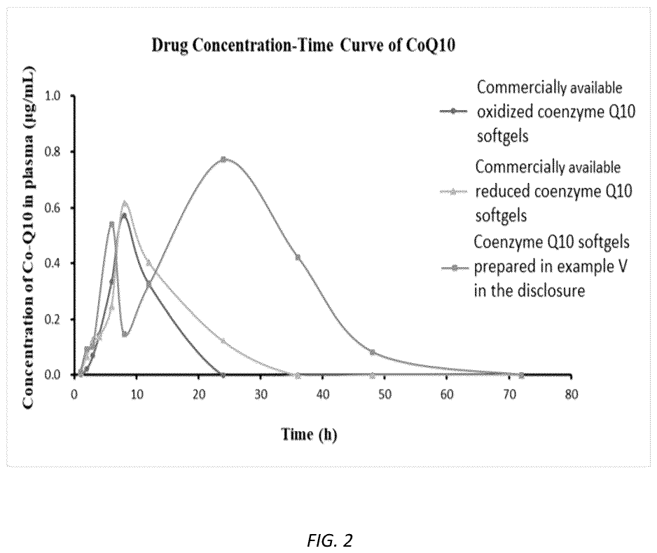

Fasting was performed before administration for 12 h (water was allowed), and the rats were allowed to take food freely in 4 h after administration. Administration was performed by gavage, 0.5 mL of blood was collected from caudal veins in 0.5, 1, 2, 3, 4, 6, 8, 12, 24, 36, 48, and 72 h after administration, was anticoagulated with heparin sodium, and was centrifuged at 4000 rpm for 10 min to take plasmas. Referring to FIG. 2, the content of the coenzyme Q10 was determined by high performance liquid chromatography, and a time-dependent changing curve of blood concentrations was drawn. An area under the curve was calculated with DAS software. In addition, the relative bioavailability was calculated by using a formula, and the obtained results are shown in the following table 3.

TABLE 3
Pharmacokinetic parameters (n = 6) of the commercially
available oxidized coenzyme Q10 softgels, commercially
available reduced coenzyme Q10 softgels, and the coenzyme
Q10 softgels disclosed by the disclosure

| | | Commercially | Commercially | Coenzyme Q10 |
|---|---|---|---|---|
| | | available | available | softgels |
| | | oxidized | reduced | prepared in |
| | | coenzyme Q10 | coenzyme Q10 | example V in |
| Parameter | Unit | softgels | softgels | the disclosure |
| AUC | Mg/L*h | 5.305 | 7.446 | 20.49 |
| CL | L/h/kg | 1.885 | 1.343 | 0.488 |
| t_{1/2} | h | 5 | 7 | 14 |
| Tmax | h | 8 | 8 | 24 |
| Cmax | Mg/L | 0.5707 | 0.6159 | 0.7718 |

It could be seen that compared with the commercially available oxidized coenzyme Q10 softgels, the bioavailability of the self-emulsifying softgels of the coenzyme Q10 (Ultra-Q10sorb) prepared in example 5 of the disclosure was increased by 4 times; and compared with the commercially available reduced coenzyme Q10 softgels, the bioavailability was increased by 3 times. Therefore, the in vivo absorption process of the drug by the self-emulsifying preparation of the coenzyme Q10 was somewhat improved.

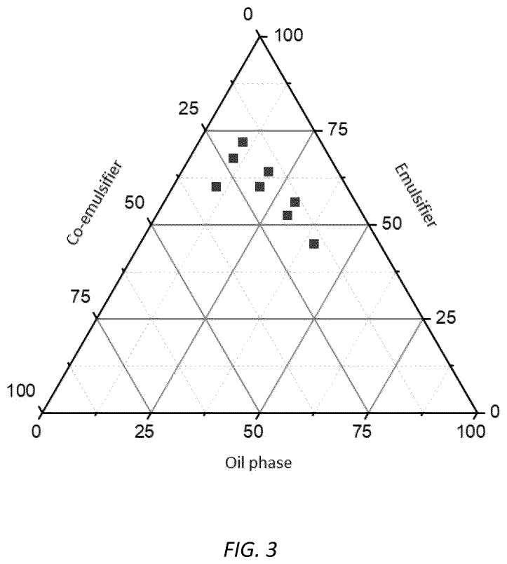

As shown in FIG. 3, accessories with better compatibility are elected by a self-emulsifying five-star evaluation method to establish the pseudo-ternary phase diagram. Mixed surfactants were respectively prepared according to the mass ratios of surfactants to cosurfactants of 1:1, 2:1, 3:1, and 4:1 based on the pseudo-ternary phase diagram, and then the mixed surfactants were mixed with the oil phase, where the mass fraction of the oil phase was 10%. A proper amount of the above micro-emulsion matrix was taken and added to deionized water, and the mixture was slightly shaken. The appearance of the liquid was observed, the mass ratio of the oil phase was improved, and the above operations were repeated. A ternary phase diagram was drawn by taking the oil phase, the surfactant, and the cosurfactant as three vertexes of the triangular phase diagram, and a self-microemulsion area was drawn at a prescription proportion with the average particle size being less than 100 nm. In the ternary phase diagram with the maximum self-microemulsion area, a proper proportion was selected as an alternative prescription.

To sum up, the self-microemulsion solution of the coenzyme Q10 obtained in the disclosure can be kept stable at relative low and high temperature ranges, the system is kept at good transparency and is free of phase splitting and crystallization and precipitation phenomena, and the emulsification is not affected as the self-microemulsion solution is stored.

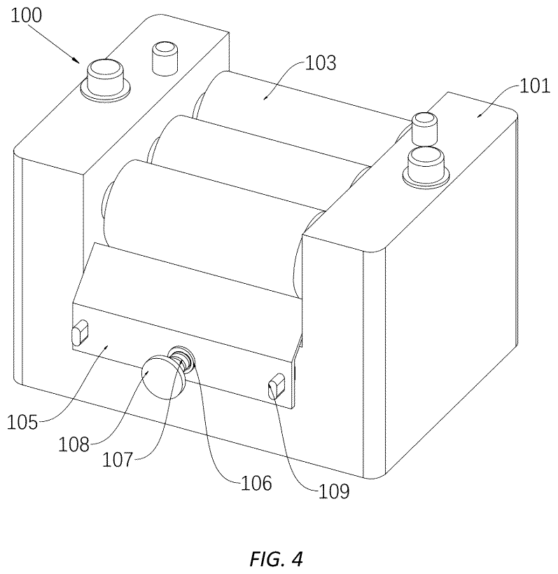

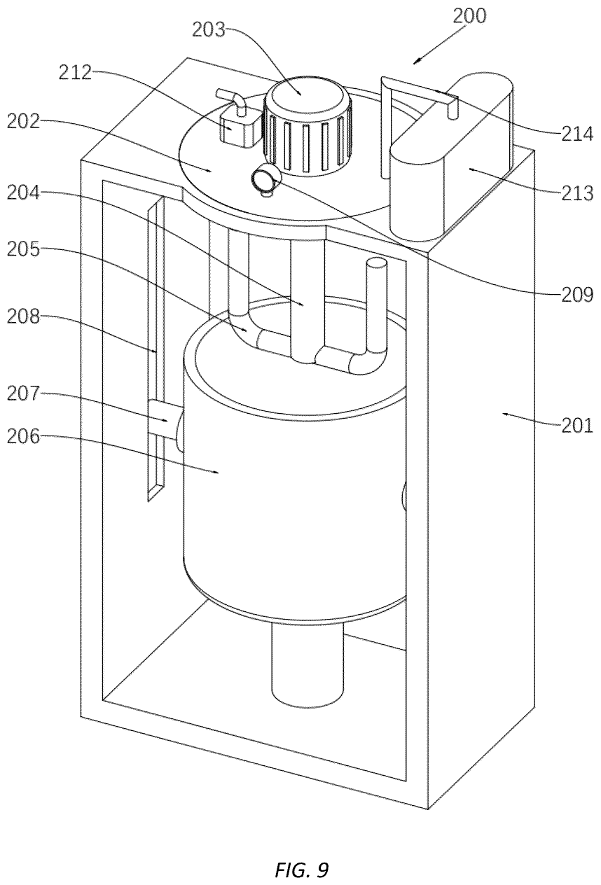

As shown in FIG. 4 and FIG. 9, the disclosure further provides a device for preparing the self-emulsifying composition of a coenzyme Q10, including a multi-roller grinder 100 and an emulsifying vacuum tank 200.

The multi-roller grinder 100 includes a frame body 101, and three rollers 103 are rotationally mounted on the frame body 101. The three rollers 103 are arranged parallel to each other, and micro gaps are reserved thereamong. A mounting cavity 102 is arranged in the frame body 101, three motors 104 are mounted in the mounting cavity 102, and power output shafts of the three motors 104 are connected to the three rollers 103, respectively. When the three motors 104 run, they drive corresponding rollers 103 to perform non-synchronous running. The rollers 103 are formed by centrifugally casting chilled hard cast alloy iron, with the surface hardness reaching HS70° or more. It is obvious that the multi-roller grinder in the embodiment has three rollers 103 and the same number of motors 104, and two, four or other number of rollers and motors may also be arranged as needed.

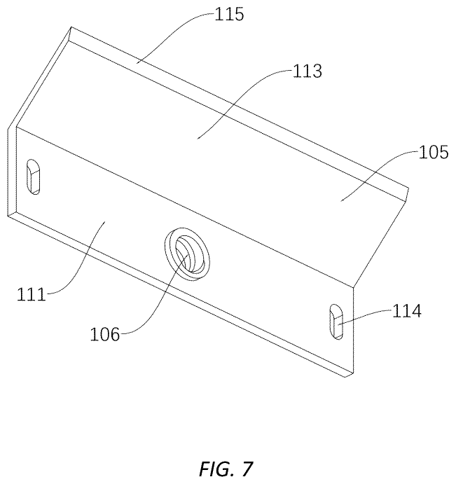

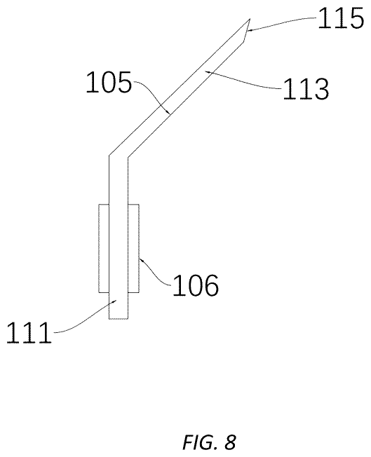

As shown in FIG. 4, FIG. 7, and FIG. 8, a shoveling apparatus is arranged on a side surface 110 of the frame body 101. The shoveling apparatus includes a shovel blade 105, a threaded barrel 106 arranged on the shovel blade 105, and a threaded rod 107 in threaded connection to the threaded barrel 106. The shovel blade 105 is provided with a vertical part 111 and an inclined part 113. The threaded barrel 106 and through holes 114 formed in both sides of the threaded barrel 106 are mounted in a penetrating manner on the vertical part 111 of the shovel blade 105. A turnplate 108 is mounted at an end of the threaded rod 107 away from the frame body 101. A threaded hole 112 and a pair of guide bars 109 fixedly mounted on both sides of the threaded hole 112 are arranged on the side surface 110 of the frame body 101. The pair of guide bars 109 penetrates through the through holes 114 in the vertical part 111 of the shovel blade 105 and is slidably connected to the shovel blade 105. An inclined surface 115 is arranged on the inclined part 113 of the shovel blade 105, the inclined surface 115 can be in contact with the rotating rollers 103 to shovel the materials attached to the peripheral surfaces of the rollers 103, i.e., to shovel the materials protruding out of the surfaces of the rollers 103.

When the multi-grinder is used, the three motors 104 are started first to drive the three rollers 103 to perform non-synchronous rotation. Then, the coenzyme Q10 material needed to be ground is placed on the rollers 103 for grinding. Thus, when the three rollers 103 rotate, a controllable and sufficient grinding effect on the coenzyme Q10 material is achieved through extrusion friction on the surfaces of the rollers 103 at different relative speeds.

When clearing is needed after the material is ground, the motors 104 are started to drive the rollers 103 to rotate. Then the turnplate 108 is rotated to drive the threaded rod 107 to rotate while enabling the threaded barrel 106 (i.e., shovel blade 105) to move toward the direction close to the frame body 101 till the inclined surface 115 of the shovel blade 105 is in contact with the rollers 103. Thus, the residual materials protruding out of the surfaces of the rollers 103 can be cleared away thoroughly.

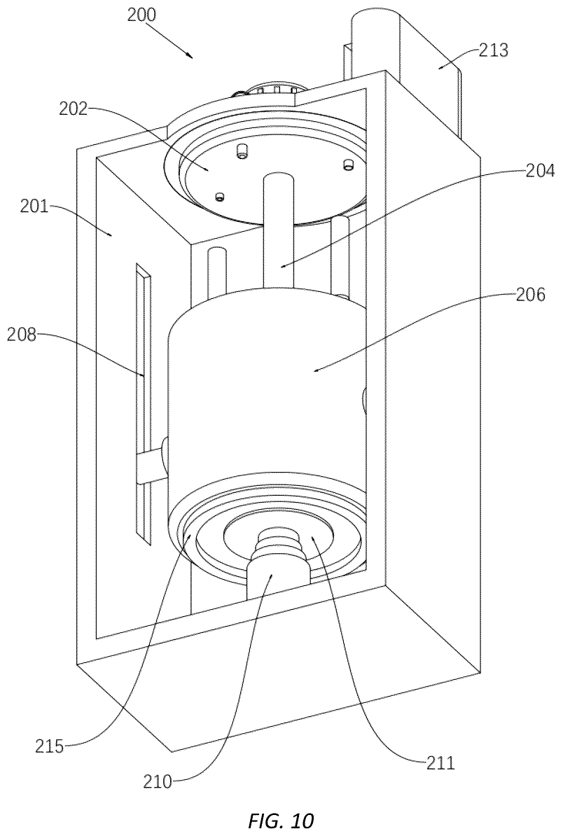

As shown in FIG. 9 to FIG. 10, the emulsifying vacuum tank 200 includes a fixed frame 201. A seal cover 202 is mounted in a penetrating manner on the top surface of the fixed frame 201, a driving motor 203 is mounted on the top surface of the seal cover 202, and a shaft lever 204 is mounted on a power output shaft of the driving motor 203. The shaft lever 204 penetrates through the seal cover 202 and extends to the lower side of the seal cover 202. A pair of stirring rods 205 is fixedly mounted at an end of the shaft lever 204. A transparent tank 206 is slidably mounted on the inner side of the fixed frame 201, and the transparent tank 206 is configured to hold the ground coenzyme Q10 and corresponding accessories. The top of the transparent tank 206 is opened, and the transparent tank 206 may be in insertion fit with the seal cover 202 for sealing. The side wall of the transparent tank 206 is made of a transparent material. A heating ring 215 is mounted on the bottom surface of the transparent tank 206, and the heating ring 215 is started to heat the bottom of the transparent tank 206, so as to heat the material located in the transparent tank 206.

As shown in FIG. 10, a multi-stage hydraulic cylinder 210 is mounted on the inner bottom surface of the fixed frame 201, a push plate 211 is mounted at an extension end of the multi-stage hydraulic cylinder 210, and the push plate 211 is fixedly connected to the bottom surface of the transparent tank 206. When the multi-stage hydraulic cylinder 210 runs, the height of the transparent tank 206 may be adjusted through the push plate 211.

As shown in FIG. 9, sliding grooves 208 are formed in inner walls of both sides of the fixed frame 201. A pair of slide bars 207 is in pivoted connection to both sides of the transparent tank 206, so that the transparent tank 206 may rotate relatively around the slide bars 207. When it is needed to take out the material in the transparent tank 206 or clean the inner wall of the transparent tank 206, the transparent tank 206 is rotated, so that the transparent tank 206 inclines toward one side. Therefore, it is convenient to pour out the material in the transparent tank 206 and it is also convenient to clean the interior of the transparent tank 206 with a tool. In addition, ends of the slide bars 207 are slidably received in the corresponding sliding grooves 208.

As shown in FIG. 9, a vacuum pump 212 and a gas-pressure meter 209 are mounted on the top surface of the seal cover 202. An air extraction end of the vacuum pump 212 extends to the lower side of the seal cover 202, and a detection end of the gas-pressure meter 209 also extends to the lower side of the seal cover 202. A gas container 213 is further mounted on the top surface of the seal cover 202. The gas container 213 is configured to store an inert gas, such as nitrogen. A gas delivery pipe 214 is mounted on the gas container 213. The gas delivery pipe 214 extends to the lower side of the seal cover 202. A switching valve (not shown in the figure) is mounted on the gas delivery pipe 214.

When the above emulsifying vacuum tank 200 is used, material inputting and closed mounting are performed first: according to the above preparation method, the coenzyme Q10 ground by the multi-roller grinder 100 and accessories in corresponding percents are added to the transparent tank 206 successively, then the multi-stage hydraulic cylinder 210 is started, so that the extension end of the multi-stage hydraulic cylinder 210 pushes the transparent tank 206 through the push plate 211, and the transparent tank 206 moves upward in the direction of the sliding grooves 208 through matched limit of the slide bars 207 and the sliding grooves 208 till the transparent tank 206 is in insertion seal with the seal cover 202.

Then the material vacuum emulsifying step is performed: the vacuum pump 212 is started first to extract air in the transparent tank 206, so that the interior of the transparent tank 206 is in a negative pressure; then the switching valve on the gas delivery pipe 214 is opened to convey the inert gas in the gas container 213 to the transparent tank 206; then, the heating ring 215 is started to heat the bottom surface of the transparent tank 206 so as to heat the material in the transparent tank 206 to a proper temperature and to be insulated; and finally, the driving motor 203 is started, so that the power output shaft of the driving motor 203 drives the shaft lever 204 to rotate to further drive the stirring rods 205 to rotate, so that the stirring rods 205 stir the material in the transparent tank 206 till the uniform and transparent coenzyme Q10 medicinal liquid is obtained by stirring. As the transparent tank 206 is filled with the inert gas, in the heating and stirring processes of the material, the material is not oxidized.

Finally, a medicinal liquid taking step is performed: the driving motor 203 is shut down, and then the vacuum pump 212 is started to extract air in the transparent tank 206 to remove bubbles in the material; after the bubbles are removed, the air pressure in the transparent tank 206 is restored to the normal air pressure, and then the multi-stage hydraulic cylinder 210 is started, so that the extension end of the multi-stage hydraulic cylinder 210 moves downward to drive the transparent tank 206 to move downward, and therefore, the transparent tank 206 is separated from the seal cover 202. Then the transparent tank 206 is rotated, so that the transparent tank 206 is in an inclined state, and therefore, it is convenient to take out the material liquid in the transparent tank 206.

It should be noted that the above preferred examples merely describe the technical concept and features of the disclosure and are intended to enable those skilled in the art can understand the content of the disclosure and implement the disclosure accordingly, rather than limiting the scope of protection of the disclosure. Equivalent changes or modifications made based on the spiritual essence of the disclosure all shall be covered within the scope of protection of the disclosure.

What is claimed is:

1. A self-emulsifying composition of a coenzyme Q10 with high bioavailability, comprising the following components in percentage by weight: 5-35% of a coenzyme Q10, 1-50% of an oil phase, 10-60% of a surfactant, 1-10% of a cosurfactant, 0-7% of an antioxidant, and 0.5-2% of pyrroloquinoline quinone disodium salt.

2. The self-emulsifying composition of a coenzyme Q10 with high bioavailability according to claim 1, comprising the following components in percentage by weight: 8-18% of a coenzyme Q10, 25-45% of an oil phase, 30-55% of a surfactant, 1-5% of a cosurfactant, 0-7% of an antioxidant, and 0.5-1.5% of pyrroloquinoline quinone disodium salt.

3. The self-emulsifying composition of a coenzyme Q10 with high bioavailability according to claim 2, comprising the following components in percentage by weight: 10-15% of a coenzyme Q10, 31-35% of an oil phase, 36-44% of a surfactant, 1-3% of a cosurfactant, 0-7% of an antioxidant, and 0.5-1% of pyrroloquinoline quinone disodium salt.

4. The self-emulsifying composition of a coenzyme Q10 with high bioavailability according to claim 1, wherein the oil phase is one or more of fish oil, medium chain triglyceride, soybean oil, olive oil, sunflower seed oil, and corn oil.

5. The self-emulsifying composition of a coenzyme Q10 with high bioavailability according to claim 1, wherein the surfactant is selected from one or more of Tween80, phospholipid, polyglycerol fatty acid ester, and mono-diglyceride.

6. The self-emulsifying composition of a coenzyme Q10 with high bioavailability according to claim 1, wherein the cosurfactant is selected from one or more of glycerinum, polyethylene glycol, and a sorbitol solution.

7. The self-emulsifying composition of a coenzyme Q10 with high bioavailability according to claim 1, wherein the antioxidant is selected from one or more of vitamin E, ascorbic acid, natural vitamin E and ester derivatives thereof, and rosemary extract.

8. The self-emulsifying composition of a coenzyme Q10 with high bioavailability according to claim 1, wherein the coenzyme Q10 is at least one of an oxidized coenzyme Q10 and a reduced coenzyme Q10.

9. A method for preparing the self-emulsifying composition of a coenzyme Q10 with high bioavailability according to claim 1, comprising the following step:
material grinding: inputting a material of a coenzyme Q10 into a multi-roller grinder for grinding and refining;
preparation of a coenzyme Q10 medicinal liquid: putting the ground coenzyme Q10 and an oil phase material in a corresponding weight percent into an emulsifying vacuum tank to be uniformly mixed, heating the mixture to 45-60° C., stirring the mixture till the coenzyme Q10 is completely dissolved, and adding the surfactant, the cosurfactant, and an accessory in corresponding weight percents to obtain the coenzyme Q10 medicinal liquid.

10. The method for preparing the self-emulsifying composition of a coenzyme Q10 with high bioavailability according to claim 9, further comprising the following step: preparing the obtained coenzyme Q10 medicinal liquid into any one of coenzyme Q10 softgels, coenzyme Q10 liquid hard capsules, a coenzyme Q10 oral solution, and coenzyme Q10 drops.

11. Use of the self-emulsifying composition of a coenzyme Q10 with high bioavailability according to claim 1 in preparation of foods, cosmetics, healthcare products or drugs.

12. A device for preparing a self-emulsifying composition of a coenzyme Q10 with high bioavailability, comprising a multi-roller grinder (100) and an emulsifying vacuum tank (200), wherein
the multi-roller grinder (100) is configured to grind a material of the self-emulsifying composition of the coenzyme Q10 and comprises a frame body (101), a plurality of rollers (103) are rotationally mounted on the frame body (101), a plurality of motors (104) are mounted on the frame body (101), the quantity of the plurality of motors (104) is consistent with the quantity of the plurality of rollers (103), and when the plurality of motors (104) run, the motors drive corresponding rollers (103) to rotate respectively and enable the plurality of rollers (103) to perform non-synchronous running to grind the material of the self-emulsifying composition of the coenzyme Q10; the multi-roller grinder (100) further comprises a shoveling apparatus (105) movably arranged on the frame body (101), and the shoveling apparatus (105) can be in contact with the rotating rollers (103) to shovel the material attached to the peripheral surfaces of the rollers (103); and
the emulsifying vacuum tank (200) is configured to self-emulsify the ground material of the self-emulsifying composition of the coenzyme Q10 and comprises a fixed frame (201), a seal cover (202) is mounted in a penetrating manner on the top surface of the fixed frame (201), a driving motor (203) is mounted on the seal cover (202), a shaft lever (204) is mounted on a power output shaft of the driving motor (203), a stirring rod (205) is mounted on the shaft lever (204), a slidable and rotatable transparent tank (206) is arranged in the fixed frame (201), and the transparent tank (206) is in insertion fit with the seal cover (202).

13. The device for preparing a self-emulsifying composition of a coenzyme Q10 according to claim 12, wherein the shoveling apparatus comprises a shovel blade (105), and the shovel blade (105) is provided with a vertical part (111) and an inclined part (113); a threaded barrel (106) and a threaded rod (107) in threaded connection to the threaded barrel (106) are mounted on the vertical part (111), a turnplate (108) is mounted at an end of the threaded rod (107) away from the frame body (101), and the inclined part (113) can be in contact with the rollers.

14. The device for preparing a self-emulsifying composition of a coenzyme Q10 according to claim 13, wherein the shoveling apparatus (105) is further provided with two guide bars (109), and the two guide bars (109) are fixedly arranged on the side surface of the frame body (101) and penetrate through the vertical part (111).

15. The device for preparing a self-emulsifying composition of a coenzyme Q10 according to claim 12, wherein a multi-stage hydraulic cylinder (210) is mounted on the inner bottom surface of the fixed frame (201), a push plate (211) is mounted at an extension end of the multi-stage hydraulic cylinder (210), and the push plate (211) is fixedly connected to the bottom surface of the transparent tank (206).

16. The device for preparing a self-emulsifying composition of a coenzyme Q10 according to claim 12, wherein sliding grooves (208) are formed on inner walls of both sides of the fixed frame (201), a pair of slide bars (207) is in pivoted connection to both sides of the transparent tank (206), and the end of each of the slide bars (207) is movably received in the corresponding one of the sliding grooves (208).

17. The device for preparing a self-emulsifying composition of a coenzyme Q10 according to claim 12, wherein a vacuum pump (212) and a gas-pressure meter (209) are mounted on the top surface of the seal cover (202), an air extraction end of the vacuum pump (212) extends to the lower side of the seal cover (202), and a detection end of the gas-pressure meter (209) also extends to the lower side of the seal cover (202); a gas container (213) is further mounted on the top surface of the seal cover (202), the gas container (213) is configured to store an inert gas, a gas delivery pipe (214) is mounted on the gas container (213), the gas delivery pipe (214) extends to the lower side of the seal cover (202), and a switching valve is mounted on the gas delivery pipe (214).
